(19) United States
(12) Patent Application Publication
(10) Pub. No.: US 2026/0345541 A1
(43) Pub. Date: Nov. 5, 2026

(54) METHODS OF TREATMENT FOR ABNORMAL REPRODUCTIVE CONDITIONS

(71) Applicant: MOREHOUSE SCHOOL OF MEDICINE, Atlanta, GA (US)

(72) Inventors: Winston E. THOMPSON, Atlanta, GA (US); Minerva GARCIA-BARRIO, Atlanta, GA (US); Luis PEREZ VILLACORTA, Atlanta, GA (US)

(21) Appl. No.: 19/666,971

(22) Filed: May 4, 2026

Related U.S. Application Data
(63) Continuation-in-part of application No. 19/655,429, filed on Apr. 22, 2026, now abandoned.
Application No. 19/655,429 is a continuation-in-part of application No. PCT/US25/29750, filed on May 16, 2025.
(63) Continuation-in-part of application No. PCT/US25/29750, filed on May 16, 2025.
(60) Provisional application No. 63/649,203, filed on May 17, 2024.

(51) Int. Cl. A61K 31/201; A61K 31/12; A61K 31/167; A61K 31/192; A61K 31/405; A61K 31/4196; A61K 31/513; A61K 31/519; A61K 31/56; A61K 31/566; A61K 31/616; A61K 38/22

(52) U.S. Cl. CPC A61K 31/201; A61K 31/12; A61K 31/167; A61K 31/192; A61K 31/405; A61K 31/4196; A61K 31/513; A61K 31/519; A61K 31/56; A61K 31/566; A61K 31/616; A61K 38/22

(57) ABSTRACT

Methods for treating an abnormal condition of the reproductive system, such as endometriosis or infertility, in a subject by administration of an effective amount of a nitrated fatty acid compound is described.

This application is a Continuation-in-Part of U.S. patent application Ser. No. 19/655,429, filed on Apr. 22, 2026, which is a Continuation-in-Part of Application No. PCT/US25/29750, filed on May 16, 2025; and a Continuation-in-Part of Application No. PCT/US25/29750, filed on May 16, 2025, which claims priority to U.S. Provisional Patent Application No. 63/649,203, filed on May 17, 2024. The entirety of the aforementioned applications is incorporated herein by reference.

This invention was made with government support under S21MD000101 awarded by the National Institutes of Health. The government has certain rights in the invention.

### FIELD

The present application is generally directed to the treatment of abnormal reproductive conditions.

### BACKGROUND

Abnormal reproductive conditions, such as endometriosis and polycystic ovary syndrome (PCOS), represent a significant and rising public health crisis. For example, Endometriosis affects about 10% of all reproductive-age women and it is one of the main causes of pelvic pain and infertility. Endometriosis accounts for about $49 billion in total US healthcare costs per year. Diagnosis and ablation of endometriotic lesions require costly albeit minimally invasive surgical techniques. Medical therapies for endometriosis consist of hormonal drugs such as progestogens, GnRH-analogues and danazol that disrupt the menstrual cycle, but these produce unwanted side-effects stemming from estrogen deficiency. Palliative pain treatment by inhibition of targeted pain receptors, such as vanilloid receptor subtypes (TRPV) and N-methyl d-aspartate (NMDA), glutamate receptors, or activation of cannabinoid receptors are novel approaches that are currently being explored. Regardless of treatment, endometriotic lesions rapidly and spontaneously recur, with reduced fecundity and ongoing pelvic pain.

While current therapeutic approaches like progestogens, GnRH-analogs, and danazol are FDA approved their effectiveness is often limited. These hormonal therapeutic approaches can hinder fertility, disrupt natural hormone levels and cause side effects like halted menstrual cycles, unwanted hair growth, postmenopausal symptoms, and osteoporosis. Alternative therapeutic strategies are geared towards drugs without hormonal side effects but with limited therapeutic efficacy, including NSAID treatment for pain. To date, no effective therapies without hormonal side effects exist. Thus, there is a major clinical need FOR novel therapeutic targets against endometriosis, pelvic inflammation, and fibrosis devoid of unwanted side effects stemming from estrogen deficiency.

Citation or identification of any document in this application is not an admission that such a document is available as prior art to the present invention.

### SUMMARY

One aspect of the present application is directed to a method for the treatment of a subject having an abnormal condition of the reproductive system, the method comprising administering to the subject an effective amount of a nitrated fatty acid compound selected from the group consisting of NO₂-cLA, CP-36, CP-45, CP-46, CP-51, and analogues/derivatives thereof.

Another aspect of the present application is directed to a method for the treatment of infertility in a subject, the method comprising administering to the subject an effective amount of a nitrated fatty acid compound selected from the group consisting of NO₂-cLA, CP-36, CP-45, CP-46, CP-51, and analogues/derivatives thereof.

Another aspect of the present application is directed to a method for the treatment of endometriosis in a subject, the method comprising administering to the subject an effective amount of a nitrated fatty acid compound selected from the group consisting of NO₂-cLA, CP-36, CP-45, CP-46, CP-51, and analogues/derivatives thereof.

One aspect of the present application is directed to a method for the treatment of a subject having a urological disorder, the method comprising administering to the subject an effective amount of a nitrated fatty acid compound selected from the group consisting of NO₂-CLA, CP-36, CP-45, CP-46, CP-51, and analogues/derivatives thereof.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings. The figures herein are for illustrative purposes only and are not necessarily drawn to scale.

FIG. 1 shows the structures of the 9-isomer form (top) and 12-isomer form (bottom) of NO₂-cLA.

FIG. 2 shows NO₂-cLA reduces endometriotic lesions in a syngeneic model of endometriosis. Peritoneal endometriosis was established using uterine fragments of PMSG-treated (3 days) EGFP female mice as donor (syngeneic model) and transplanted via i.p. injection into C57BL/6J female recipients (WT). Sham consisted of PBS i.p. injection (1 ml). Mice were treated with NO₂-cLA (2.5 mg/mice, s.c.) or vehicle (1% EtOH in PBS) daily for 28 days. FIG. 2, panel A) Donor-derived lesions were demonstrated using an AmiHTX imager in fluorescence mode (EGFP). FIG. 2, panel B) Lesions were quantified using Aura 4.0 imaging software. Data are plotted as box and whiskers. **p<01, ***p<. 001 vs. Sham; \hat{ }\hat{ }\hat{ }p<. 001, vs. PBS. n=4. ANOVA followed by Bonferroni post hoc test.

FIG. 3 shows endometriosis is reduced in STING KO mice. FIG. 3, panel A) Peritoneal endometriosis was established by injecting minced PMSG-treated EGFP uterine tissue into the peritoneum of STING KO and WT female mice and lesions were allowed to develop for 28 days. FIG. 3, panel B) Endometriotic lesions were visualized via in vivo imaging and quantified. Data is plotted as box and whiskers from minimum to maximum values. Data was analyzed by ANOVA followed by Bonferroni post hoc test, n=8. ns: not significant, ***p<0.001; ****p<0.0001. FIG. 3, panel C) Macroscopic quantification of endometriotic lesions was done at end-point analysis using a digital caliper. Data are expressed as diameter size (in mm). Mice with no observable lesions were included in the analysis (considered as zero). Data are shown as violin plots showing all points. Welch's t-test **p=0.0014.

FIG. 4 shows in vitro therapeutic index based on inhibition of type I IFN release plotted against level of cytotoxicity. For type I IFN release, SAVI fibroblasts were pre-treated with indicated nitroalkene compounds (2, 5, 10 and 15 μM) for 2 h, followed by cGAMP stimulation (4 μg/mL) for 24 h. The cytotoxicity was measured by MTT assay, SAVI fibroblasts were treated with indicated nitroalkene compounds in different concentrations (2, 5, 10, 15, 20, 30, 50, 70 and 100 μM) for 24 h. Data of the therapeutic index represent the mean±SEM of two (IFN) and three (MTT) replicates. The type I IFN release is normalized to levels reached with cGAMP treatment, expressed as percentage. The cytotoxicity is normalized to untreated cells, expressed as percentage viability. The dotted line denotes 75% inhibition of the type I IFN response induced by cGAMP. The EC₅₀ value is determined by fitting the curve with nonlinear regression. The asterisk and bold axes indicate the lead compounds with a high in vitro therapeutical index, due to high potency to inhibit type I IFN production with minimal cytotoxicity.

FIG. 5 shows efficient STING inhibition and low toxicity by CP-36 in SAVI patients-derived fibroblast and murine BMDMs. FIG. 5, panel A) STING-dependent inhibition was measured by CXCL-10 release induced by cGAMP and cytotoxicity measurements MTT assays. Several conjugated nitroalkene compounds inhibited CXCL-10 release, while showing minimal cytotoxicity including CP-36, whereas other structurally similar NO₂-FA (i.e. NO₂-EA) had no effect. FIG. 5, panel B) Specificity of CP36 was tested in BMDMs upon stimulation of cGAMP (dissolved in Lipofectamine 2000-Lipo). CP-36 dose-dependently inhibits IFNB expression whereas equimolar concentration of reduced (inactive) CP36 (R CP36) has no effect. Data are shown as mean±SEM from BMDMs from 6 independent mice. **p≤0.01, vs. control; \hat{ }\hat{ }p≤0.01, vs cGAMP, One-way ANOVA, Bonferroni post hoc. FIG. 5, panel C) representative western blot analysis of STING inhibition by CP36 (pSTING) or pIRF3.

FIG. 6 shows in vitro therapeutic index of CP-36 in 12Z endometriotic epithelial cells. FIG. 6, panel A) In vitro therapeutic index based on inhibition of IFNβ expression plotted against level of cytotoxicity. IFNβ mRNA expression, endometriotic epithelial cells (12Z) were pre-treated with indicated CP-36 (1, 2, 4 and 8 μM) for 2 h, followed by cGAMP stimulation (4 μg/mL) for 4 h. Cytotoxicity in 12Z cells was measured by MTT assay, upon treatment with CP-36 in different concentrations (1, 2, 4, 8, 16, 32, 64, 128 μM) for 24 h. IFNb mRNA expression is normalized to levels reached with cGAMP, expressed as percentage. Cytotoxicity was normalized to untreated cells. Data of the therapeutic index represents the mean±SEM of 3 replicates. FIG. 6, panel B) 12Z cells were grown in DMEM/F12 supplemented with 10% FBS, synchronized by 2% FBS overnight and treated as follows: cells were treated with cGAMP vehicle (Lipo3000) and treated with CP-36 or R-CP-36, prior to stimulation with STING agonist cGAMP (4 ng/ml) for 6 h. Dosedependent inhibition of viperin mRNA expression by CP-36 (2 μM, 5 μM) or R-CP-36, (n=4 biological replicates), One-way ANOVA, Bonferroni post hoc test, *p≤0.05, **p≤0.01; **p≤0.01; ***p≤0.001.

FIG. 7 shows oral CP-36 is readily bioavailable and metabolized and efficiently inhibits STING signaling I. FIG. 7, panel A) Steady-state plasma levels of CP36 at 6 h per o.a. and its dehydro-CP-36 derivative (metabolite) FIG. 7, panel B) upon oral delivery of 40 mg/kg and 60 mg/kg. n=10 **p<0.01, vs. control or cGAMP; FIG. 7, panel C) Western blot analysis of STING target genes (viperin, ISG15) and inflammatory pathways (STAT1) is efficiently reduced by CP-36. FIG. 7, panel D) Inhibition of IFNβ expression in the liver by CP-36 analyzed by qPCR. n=10. **p≤0.01, vs. control; \hat{ }p≤0.05, vs cGAMP, One-way ANOVA, Bonferroni post hoc test.

FIG. 8 shows expression of STING in human eutopic endometrium and endometriotic lesions. Immunofluorescent localization of STING and EpCAM performed in TMA sections from endometriosis of endometrium, ovary and peritoneal tissues. Sections were counter stained with 4′,6-diamidino-2-phenylindole (DAPI) to visualize all nuclei. Colocalization of STING and EpCAM is observed in normal and endometriotic endometrium, primarily at the epithelial layer of the grands whereas STING stromal expression was weaker FIG. 8, panel A) and in ovarian and peritoneal endometriotic lesions from humans FIG. 8, panel B) whereas STING stromal expression was weaker compared to epithelial in normal and endometriotic tissue. Size bar=200 μm.

FIG. 9 shows NO₂-cLA inhibits STING activation in endometriotic stromal cells. EESC were obtained from eutopic biopsies of an endometriosis patient in the proliferative phase at Emory Midtown Hospital. NESCs were obtained from recruited parous woman with regular menstrual cycles. Cells were grown in DMEM/F12 10% FBS, synchronized 2% FBS overnight and treated as follows: FIG. 9, panel C) vehicle (Lipo3000), NO₂-CLA (2 μM) or cLA, prior stimulation with cGAMP (4 ng/mL) for 6 h. Dose-dependent inhibition of CCL2 (FIG. 9, panel A) and IL6 (FIG. 9, panel B) mRNA expression by NO₂-CLA (1 μM, 2 μM), 3 h poststimulation with cGAMP. (FIG. 9, panel C) Inhibition of STING activity (p-STING/tSTING) by NO₂-cLA but not cLA (2 μM), 6 h after cGAMP, by western blot. Data shown as mean±s.e.m (n=3 biological replicates), *p≤0.05, **p≤0.01 vs. Control; ++p≤0.01 vs cGAMP; \hat{ }\hat{ }p≤0.01 vs. cLA. ANOVA, Bonferroni post-hoc.

FIG. 10 shows generation of STING C88A/C91A knock-in (KI) mice, and its Reproductive behavior upon oral CP-36 treatment. FIG. 10, panel A) Schematic depiction of the STING genomic region with the key elements for the KI strategy via homologous recombination. Pink bar: knock-in oligo with C88A and C91A substitutions; green arrows: guide RNAs designed using ZiFiT software. The 159 bases-long knock-in oligo encoded both mutations and the proto-spacer adjacent motif (PAM) sequence. FIG. 10, panel B) Genotyping sequencing results and FIG. 10, panel C) sequence alignment confirming C88A/C91A KI mutation in the founder (F0). The F0 was backcrossed to C57BL6 to ensure that the model is void of off-target genomic effects (over 10 crosses). FIG. 10, panel D) Ratios of pups from STING KI heterozygous crosses showing that homozygous mice are born at Mendelian ratios (Exp: expected, Obs: observed). FIG. 10, panel E) STING KI homozygous and STING KO were mated and confirmed pregnant females (via plug visualization) treated with CP-36, R-CP-36 or PEG (vehicle) orally (5 mg/kg/day) during gestation. Viable pups per litter were counted.

FIG. 11 Shows click chemistry to detect specific covalent adduction of 3 μM Az-NO₂-OA to PPARγ Cys 285 for 3 h.

FIG. 12 shows oral NO₂-FA administration reduces syngeneic endometriotic lesions. FIG. 12, panel A) For oral delivery experiments, mice were randomized for treatment of either NO₂-cLA, CLA (5 mg/kg/day), or vehicle (PEG) for 28 days. FIG. 12, panel B) Representative MS tracing and quantitation of NO₂-cLA and its metabolite (DH-NO₂-cLA) levels in plasma and peritoneal fluid (P.F). FIG. 12, panel C) Representative images of EGFP-derived lesions in response to each treatment. FIG. 12, panel D) EGFP emission at the peritoneal cavity was quantified using Aura 4.0 imaging. Data is plotted as box and whiskers from min. to max. values. Data analyzed by ANOVA followed by Bonferroni post hoc test. ns: not significant, **p<0.01; *p<0.05. FIG. 12, panel E) Macroscopic quantification of endometriotic lesions was done at end-point analysis (28 days) using a digital caliper. Data is expressed as diameter size (in mm). Animals with no observable lesions were included in the analysis (considered as zero). Data is shown as violin plots showing all points. ****p<0.0001, *p<0.05.

FIG. 13 shows STING KO mice display reduced levels of surrogate markers of pain. FIG. 13, panel A) IFNβ and FIG. 13, panel B) Fractalkine levels in plasma and peritoneal fluid assessed by Multiplex assays. ****p<0.0001. nd: not detected. ns not significant. ANOVA followed by Bonferroni post hoc test.

FIG. 14 shows loss-of-STING function in the uterine fragments transplanted in WT syngeneic reduces endometriosis lesion formation. (FIG. 14, panel A) Scheme depicting a reverse genetic approach in which endometriosis was established using WT and STING KO mice as donors transplanted in WT syngeneic mice. (FIG. 14, panel B) Lesion diameter was measured at end-point analysis (28 days) via digital caliper, demonstrate that lesions derived from STING KO mice are significantly reduced compared to WT. Welch's t-test **p=0.0016. (FIG. 14, panel C) Pro-inflammatory CXCL10/IP-10 expression levels in plasma and peritoneal fluid were quantified by Multiplex assays. Data points were plotted as violin plots. ns: not significant.

FIG. 15 shows a graphic of prevention of endometriosis progression. Early stage treatment with CP-36.

FIG. 16 shows a graphic of regression of endometriosis. Late stage treatment with CP-36.

### DETAILED DESCRIPTION

Reference will be made in detail to certain aspects and exemplary embodiments of the application, illustrating examples in the accompanying structures and figures. The aspects of the application will be described in conjunction with the exemplary embodiments, including methods, materials and examples, such description is non-limiting and the scope of the application is intended to encompass all equivalents, alternatives, and modifications, either generally known, or incorporated here. Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present application. The described aspects and embodiments of the application are not limited to the methods and materials described.

All publications and patents cited in this specification are cited to disclose and describe the methods and/or materials in connection with which the publications are cited. All such publications and patents are herein incorporated by reference as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’,” where ‘x’ and ‘y’ are numerical values, includes “about ‘x’ to about ‘y’.”

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

The recitation of numerical ranges by endpoints includes all numbers and fractions subsumed within the respective ranges, as well as the recited endpoints.

### DEFINITIONS

Unless defined otherwise, technical and scientific terms used herein have the same meaning as commonly understood by one of ordinary skill in the art to which this disclosure pertains.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, “abnormal reproductive condition” includes, but is not limited to, pelvic inflammatory disease (PID), endometriosis, infertility (including, but not limited to, chronic infertility syndrome, poor implantation), polycystic ovary syndrome (PCOS), ovarian cancer, ovarian cysts and fibromas, uterine fibroids, uterine cancer, adenomyosis, cervical cancer, uterine prolapse, menstrual disorders (including, but not limited to, amenorrhea, dysmenorrhea, menorrhagia), low sperm count, erectile dysfunction, and testicular cancer.

As used herein, the term “an effective amount” means an amount necessary to achieve a selected result.

As used herein, the term “analgesic” refers to agents, compounds or drugs used to relieve pain and inclusive of anti-inflammatory compounds. Exemplary analgesic and/or anti-inflammatory agents, compounds or drugs include, but are not limited to, the following substances: non-steroidal anti-inflammatory drugs (NSAIDs), salicylates, aspirin, salicylic acid, methyl salicylate, diflunisal, salsalate, olsalazine, sulfasalazine, para-aminophenol derivatives, acetanilide, acetaminophen, phenacetin, fenamates, mefenamic acid, meclofenamate, sodium meclofenamate, heteroaryl acetic acid derivatives, tolmetin, ketorolac, diclofenac, propionic acid derivatives, ibuprofen, naproxen sodium, naproxen, fenoprofen, ketoprofen, flurbiprofen, oxaprozin; enolic acids, oxicam derivatives, piroxicam, meloxicam, tenoxicam, ampiroxicam, droxicam, pivoxicam, pyrazolon derivatives, phenylbutazone, oxyphenbutazone, antipyrine, aminopyrine, dipyrone, coxibs, celecoxib, rofecoxib, nabumetone, apazone, indomethacin, sulindac, etodolac, isobutylphenyl propionic acid, lumiracoxib, etoricoxib, parecoxib, valdecoxib, tiracoxib, etodolac, darbufelone, dexketoprofen, aceclofenac, licofelone, bromfenac, loxoprofen, pranoprofen, piroxicam, nimesulide, cizolirine, 3-formylamino-7-methylsulfonylamino-6-phenoxy-4H-1-benzopyran-4-one, meloxicam, lornoxicam, d-indobufen, mofezolac, amtolmetin, pranoprofen, tolfenamic acid, flurbiprofen, suprofen, oxaprozin, zaltoprofen, alminoprofen, tiaprofenic acid, pharmacological salts thereof, hydrates thereof, and solvates thereof.

As used herein, the term “analogue” refers to a compound which comprises a chemically modified form of a specific compound or class thereof, and which maintains the pharmaceutical and/or pharmacological activities characteristic of said compound or class.

As used herein, a “biological sample” refers to a sample obtained from, made by, secreted by, excreted by, or otherwise containing part of or from a biologic entity. A biologic sample can contain whole cells and/or live cells and/or cell debris, and/or cell products, and/or virus particles. The biological sample can contain (or be derived from) a “bodily fluid.” The biological sample can be obtained from an environment (e.g., water source, soil, air, and the like). Such samples are also referred to herein as environmental samples. As used herein “bodily fluid” refers to any non-solid excretion, secretion, or other fluid present in an organism and includes, without limitation unless otherwise specified or is apparent from the description herein, amniotic fluid, aqueous humor, vitreous humor, bile, blood or component thereof (e.g. plasma, serum, etc.), breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from an organism, for example by puncture, or other collecting or sampling procedures.

As used herein, the term “blood disorder” refers to conditions that impair the function or production of blood cells, platelets, lymph fluid, blood plasma, and/or clotting proteins. Exemplary blood disorders include, but are not limited to, myelofibrosis, eosinophilic granulomatosis, and granulomatosis with polyangiitis.

As used herein, “culturing” can refer to maintaining cells under conditions in which they can proliferate and avoid senescence as a group of cells. “Culturing” can also include conditions in which the cells also or alternatively differentiate. Culturing can include one or more steps or conditions, and include in one or more steps passaging, transfer of cells, media changing, incubation temperature changes, atmospheric gas changes, and/or the like.

As used herein, the term “derivative” refers to a chemically modified compound wherein the modification is considered routine by the ordinary skilled chemist, such as an ester or an amide of an acid, protecting groups, such as a benzyl group for an alcohol or thiol, and tert-butoxycarbonyl group for an amine.

“Extended-release,” also known as sustained-release (SR), sustained-action (SA), time-release (TR), controlled-release (CR), modified release (MR), or continuous-release (CR), is a mechanism used in medicine tablets or capsules to dissolve slowly and release the active ingredient over time. The advantages of extended-release tablets or capsules are that they can often be taken less frequently than immediate-release formulations of the same drug, and that they keep steadier levels of the drug in the bloodstream, thus extending the duration of the drug action and lowering the peak amount of drug in the bloodstream.

As used herein, the term “NO₂-cLA” refers to both (or either of) the 9-isomer form ((9E,11E)-9-nitro-9,11-octadecadienoic acid) and 12-isomer form ((9E,11E)-12-nitro-9,11-octadecadienoic acid) of “nitro-conjugated linoleic acid.”

The term “optional” or “optionally” means that the subsequent described event, circumstance or substituent may or may not occur, and that the description includes instances where the event or circumstance occurs and instances where it does not.

As used herein, the phrase “pharmaceutically acceptable” is used with reference to compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluensulfonic, methanesulfonic, ethane disulfonic, oxalic, isethionic, and the like.

The terms “subject,” “patient,” and “individual,” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed by such terms. Unless otherwise specified, a “subject,” “patient,” or “individual” refers to both biological male and female mammals.

As used herein, the term “skin disease” refers to conditions affecting the skin's texture, color, and/or appearance. Exemplary skin diseases include, but are not limited to, eczema, lupus erythematosus, psoriasis, skin cancers, and vitiligo.

The appropriate dosage (“therapeutically effective amount”) of the active agent(s) in the immediate-release component or the extended-release component will depend, for example, on the severity and course of the condition, the mode of administration, the bioavailability of the particular agent(s), the age and weight of the patient, the patient's clinical history and response to the active agent(s), discretion of the physician, etc.

As used herein, the term “urological disorder” refers to disorders, conditions, and diseases of the female and male urinary tract (kidneys, ureters, bladder, ureter, prostate). Exemplary urological disorders include, but are not limited to, benign prostatic hyperplasia (BPH), bladder cancer, cystitis (including interstitial cystitis), erectile dysfunction (ED), hematuria, hydronephrosis, interstitial cystitis (painful bladder syndrome), kidney stones (calculus of kidney), lupus nephritis, nephritis, obstructive uropathy, orchitis, overactive bladder (OAB), prostate cancer, prostatitis, ureteral inflammation, urethral inflammation, urethral stricture, urinary tract infection (UTI), urinary incontinence, and varicocele.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention. For example, in the appended claims, any of the claimed embodiments can be used in any combination.

Emerging evidence from endometriosis patients suggests that stimulator of interferon (IFN) genes (STING) is upregulated in endometriosis lesions. Type I and II IFN are associated with more advanced stages of endometriosis (Stage III and IV) in the clinical settings, independently of the menstrual cycle and hormonal status. Indeed, STING-dependent IFN related genes, are gaining momentum as novel stromal markers of endometriosis. Nitro-fatty acids (NO₂-FA) have emerged as potent anti-inflammatory and anti-fibrotic agents. These bioactive lipids exert protective roles in numerous experimental models of inflammation, metabolism and fibrosis. Successfully completed phase I clinical trials have addressed the safety and pharmacokinetics of nitro-oleic acid (NO₂-OA) and phase II trials are currently addressing its therapeutic potential. Like NO₂-OA, NO₂-cLA is basally present in the plasma of healthy individuals. Using targeted lipidomics, residues Cys88 and Cys91 of STING have been identified as NO₂-FA targets. Recent R&D efforts identified New Chemical Entities (NCEs) with high efficacy on STING inhibition and negligible toxicity upon oral administration. Thus, considering the unique pathogenesis of endometriosis, including endometrial inflammation and fibrosis along with the well-established anti-inflammatory properties of NO₂-FA, it is plausible that STING inhibition by our NCEs will have therapeutic potential against endometriosis. The present studies establish CP-36, the second generation of nitroalkenes as STING inhibitors, as a novel drug therapy against endometriosis. These studies provide evidence that: 1) STING is overexpressed in human ovarian and peritoneal endometriosis and preferentially expressed in the epithelial eutopic endometrium of endometriotic patients. 2) NO₂-FA administration protects against endometriosis regardless of the route of administration in a syngeneic model of endometriosis. 3) Loss-of-STING function protects against experimental endometriosis. 3) Novel NCEs (e.g., CP-36) with improved STING affinity (IC50) and low toxicity in vivo and in endometriotic epithelial cells are readily bioavailable as an oral therapy against endometriosis. Based on these premises, the studies employ unique mouse models, STING C88A/C91A knock-in, and STING KO to assess STING function in epithelial and stromal cells and to test the therapeutic specificity of CP-36 as STING inhibitor in syngeneic peritoneal endometriosis. The present application provides orally bioavailable STING inhibitors protect against endometriosis and associated pain by inhibiting inflammation and fibrosis.

NO₂-FAs are generated during inflammation and digestion through non-enzymatic reactions of unsaturated fatty acids with nitrogen dioxide (NO₂), yielding an array of electrophilic NO₂-FAs with unique biochemical and signaling properties, including activation of PPARγ, Nrf2, heat-shock factor (HSP), amongst other targets. Nitro-oleic acid (NO₂-OA) exerts protective roles in numerous experimental models of inflammation, imbalanced lipid metabolism and fibrosis. The present inventors have pioneered studies on the role of NO₂-FA in innate immune responses demonstrating the anti-inflammatory role of both NO₂-OA and NO₂-cLA in well-established models of acute and chronic inflammatory responses sterile inflammation. Over the last 10 years, the functional characterization of NO₂-FAs have revealed clinically relevant protection in a number of cardiovascular and metabolic experimental models, including multi-organ injury, colitis, atherosclerosis, systemic and pulmonary arterial hypertension (PAH), atrial fibrillation and myocardial fibrosis; angioplasty-induced restenosis, cardiac ischemia-reperfusion (I/R) injury, and our recent work on its therapeutic potential in fatty liver disease. Most of these studies used subcutaneously implanted pumps with NO₂-OA as a prototypic electrophilic NO₂-FA. In this manner, a steady state endogenous plasma concentration of NO₂-OA reached 20 nM, clearly demonstrating a pharmacological efficacy of NO₂-OA in the nanomolar range. The safety and pharmacokinetics of NO₂-OA have been clinically examined in four successfully completed phase I trials (NCT02127190, NCT02248051, NCT02460146, NCT02313064) and its therapeutic potential is currently being evaluated in phase II clinical trials for the treatment pulmonary fibrosis in asthmatics (NCT03762395).

The present application defines CP-36 as a bona fide STING inhibitor in endometriosis. Site-directed mutagenesis approaches aligned with “click chemistry” in these studies define STING residues directly engaged in CP-36 binding both in vitro and in vivo, and in the uterus in syngeneic endometriosis. 2) In vitro analysis of inflammation, fibrosis, and angiogenesis in response to CP-36 are defined on epithelial and stromal cells isolated from wild-type, STING KI and STING KO mice.

This application further establishes that STING inhibition by oral delivery of CP-36 reduces endometriosis. Syngeneic models of endometriosis are utilized to: 1) demonstrate that STING hyperactivation in endometriotic cells contributes to endometriosis engraftment in the peritoneum; 2) establish that STING inhibition by orally delivered CP-36 reduces endometrial lesion formation by inhibiting inflammation, fibrosis, angiogenesis and immune cell infiltration in the peritoneum; and 3) establish CP-36 as a novel drug therapy by assessing regression of established endometriosis via inhibition of inflammation and fibrosis, and alleviation of pain.

### Treatment

One aspect of the present application is directed to a method for the treatment of a subject having an abnormal condition of the reproductive system, the method comprising administering to the subject an effective amount of one or more nitrated fatty acid compounds selected from the group consisting of NO₂-cLA, CP-36, CP-45, CP-46, CP-51, and analogues/derivatives thereof. In some embodiments, NO₂-cLA consists of the 12-isomer form. In some embodiments, NO₂-cLA consists of the 9-isomer form. In some embodiments, NO₂-cLA comprises both the 12-isomer form and the 9-isomer form.

In some embodiments, the abnormal condition of the reproductive system is a disease of the female reproductive system including, but not limited to, endometriosis, uterine fibroids, a chronic infertility disease (such as poor implantation), adenomyosis, and pelvic inflammatory disease.

Another aspect of the present application is directed to a method for the treatment of infertility in a subject, the method comprising administering to the subject an effective amount of one or more nitrated fatty acid compounds selected from the group consisting of NO₂-cLA, CP-36, CP-45, CP-46, CP-51, and analogues/derivatives thereof. In some embodiments, NO₂-cLA consists of the 12-isomer form. In some embodiments, NO₂-cLA consists of the 9-isomer form. In some embodiments, NO₂-cLA comprises both the 12-isomer form and the 9-isomer form.

Another aspect of the present application is directed to a method for the treatment of endometriosis in a subject, the method comprising administering to the subject an effective amount of one or more nitrated fatty acid compounds selected from the group consisting of NO₂-CLA, CP-36, CP-45, CP-46, CP-51, and analogues/derivatives thereof. In some embodiments, NO₂-cLA consists of the 12-isomer form. In some embodiments, NO₂-cLA consists of the 9-isomer form. In some embodiments, NO₂-cLA comprises both the 12-isomer form and the 9-isomer form.

Another aspect of the present application is directed to a method for treating urological disorders and/or diseases of the male reproductive system in a subject, the method comprising administering to the subject an effective amount of one or more nitrated fatty acid compounds selected from the group consisting of NO₂-CLA, CP-36, CP-45, CP-46, CP-51, and analogues/derivatives thereof. In some embodiments, NO₂-cLA consists of the 12-isomer form. In some embodiments, NO₂-cLA consists of the 9-isomer form. In some embodiments, NO₂-CLA comprises both the 12-isomer form and the 9-isomer form. Exemplary urological disorders and/or diseases of the male reproductive system include, but are not limited to prostatitis, orchitis, obstructive uropathy, hydronephrosis, bladder cancer, interstitial cystitis, urinary tract infection, urethral inflammation, and ureteral inflammation.

Another aspect of the present application is directed to a method for treating a disease selected from the group consisting of blood disorders in a subject, the method comprising administering to the subject an effective amount of one or more nitrated fatty acid compounds selected from the group consisting of NO₂-cLA, CP-36, CP-45, CP-46, CP-51, and analogues/derivatives thereof. In some embodiments, NO₂-cLA consists of the 12-isomer form. In some embodiments, NO₂-cLA consists of the 9-isomer form. In some embodiments, NO₂-cLA comprises both the 12-isomer form and the 9-isomer form. Exemplary blood disorders include, but are not limited to, myelofibrosis, eosinophilic granulomatosis, and granulomatosis with polyangiitis.

Another aspect of the present application is directed to a method for treating a skin disease in a subject, the method comprising administering to the subject an effective amount of one or more nitrated fatty acid compounds selected from the group consisting of NO₂-cLA, CP-36, CP-45, CP-46, CP-51, and analogues/derivatives thereof. In some embodiments, NO₂-cLA consists of the 12-isomer form. In some embodiments, NO₂-cLA consists of the 9-isomer form. In some embodiments, NO₂-cLA comprises both the 12-isomer form and the 9-isomer form. Exemplary skin diseases include, but are not limited to, eczema, psoriasis, skin cancers, and vitiligo.

In some embodiments, the nitrated fatty acid compound is the 12-nitrated isomer of NO₂-cLA ((9E,11E)-12-nitro-9,11-octadecadienoic acid):

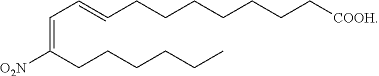 [SMILES: CCCCCC/C(=C\C=C\CCCCCCCC(=O)O)[N+](=O)[O-]]

In some embodiments, the nitrated fatty acid compound is the 9-nitrated isomer of NO₂-cLA ((9E,11E)-9-nitro-9,11-octadecadienoic acid):

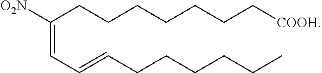 [SMILES: CCCCCC/C=C/C=C(\CCCCCCCC(=O)O)[N+](=O)[O-]]

In some embodiments, the nitrated fatty acid compound is CP-36 ((E)-3-(3-(2-nitrohept-1-en-1-yl)phenyl) propanoic acid):

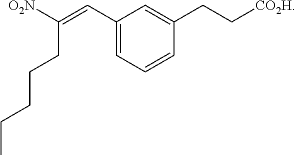 [SMILES: CCCCC/C(=C\c1cccc(CCC(=O)O)c1)[N+](=O)[O-]]

In some embodiments, the nitrated fatty acid compound is CP-45 (methyl (E)-4-nitro-5-(p-tolyl) pent-4-enoate):

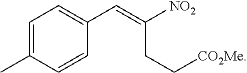 [SMILES: COC(=O)CC/C(=C\c1ccc(C)cc1)[N+](=O)[O-]]

In some embodiments, the nitrated fatty acid compound is CP-46 (allyl (E)-6-nitro-7-(p-tolyl) hept-6-enoate):

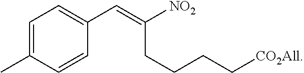

In some embodiments, the nitrated fatty acid compound is CP-51 (ethyl (E)-7-nitro-7-phenylhept-6-enoate):

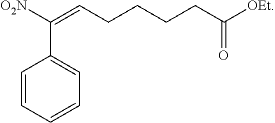 [SMILES: CCOC(=O)CCCC/C=C(\c1ccccc1)[N+](=O)[O-]]

In some embodiments, an agent of the present application is administered orally. Suitable compositions for oral administration include, but are not limited to: tablets, coated tablets, gelcaps, dragées, capsules, powders, granulates and soluble tablets, and liquid forms, for example, suspensions, dispersions or solutions.

In some embodiments, an agent of the present application is administered transdermally.

In some embodiments, an agent of the present application is administered intravenously, intradermally, subcutaneously, or intraperitoneally.

In some embodiments, an agent of the present application is administered as a vaginal suppository.

In some embodiments, a therapeutically effective amount of an agent of the present application is administered daily in a single dose. In some embodiments, a therapeutically effective amount of an agent of the present application is administered 2, 3, 4, 6, 8, or 12 times per day. In some embodiments, a therapeutically effective amount of an agent of the present application is administered once every 2, 3, 4, 5, 6, 7, or 14 days. In some embodiments, a therapeutically effective amount of an agent of the present application is administered monthly.

As a general proposition, the therapeutically effective amount of the agent is administered in the range of about 100 μg/kg body weight to about 100 mg/kg body weight whether by one or more administrations. In some embodiments, the range of each active agent administered daily in a single dose or in multiple does is from about 100 μg/kg body weight to about 50 mg/kg body weight, 100 μg/kg body weight to about 10 mg/kg body weight, 100 μg/kg body weight to about 1 mg/kg body weight, 100 μg/kg body weight to about 10 mg/kg body weight, 500 μg/kg body weight to about 100 mg/kg body weight, 500 μg/kg body weight to about 50 mg/kg body weight, 500 μg/kg body weight to about 5 mg/kg body weight, 1 mg/kg body weight to about 100 mg/kg body weight, 1 mg/kg body weight to about 50 mg/kg body weight, 1 mg/kg body weight to about 10 mg/kg body weight, 5 mg/kg body weight/dose to about 100 mg/kg body weight, 5 mg/kg body weight/dose to about 50 mg/kg body weight, 10 mg/kg body weight to about 100 mg/kg body weight, and 10 mg/kg body weight to about 50 mg/kg body weight.

In some embodiments, a second therapeutic agent is administered to the subject.

In some embodiments, the nitrated fatty acid compound is administered before, at the same time as, or after the second therapeutic agent.

In some embodiments, the nitrated fatty acid compound and the second therapeutic agent are in the same pharmaceutical composition.

In some embodiments, the nitrated fatty acid compound and the second therapeutic agent are in separate pharmaceutical compositions.

In some embodiments, the second therapeutic agent comprises one or more analgesic agents. In some embodiments, the one or more analgesic agents are selected from the group consisting of aspirin, ibuprofen, naproxen, naproxen sodium, indomethacin, nabumetone, and acetaminophen.

In some embodiments, the second therapeutic agent comprises one or more hormonal contraceptives.

In some embodiments, the second therapeutic agent comprises one or more progestins. In some embodiments, the one or more progestins are selected from the group consisting of norethindrone, norethindrone acetate, ethynodiol diacetate, levonorgestrel, desogestrel, gestodene, norgestimate, drospirenone, and medroxyprogesterone acetate.

In some embodiments, the second therapeutic agent comprises one or more GnRH agonists. In some embodiments, the one or more GnRH agonists are selected from the group consisting of leuprorelin or goserelin.

In some embodiments, the second therapeutic agent comprises one or more GnRH antagonists. In some embodiments, the one or more GnRH antagonists are selected from the group consisting of elagolix, relugolix, and linzagolix.

In some embodiments, the second therapeutic agent comprises one or more aromatase inhibitors. In some embodiments, the one or more aromatase inhibitors are selected from the group consisting of anastrozole, letrozole, and exemestane.

In some embodiments, the second therapeutic agent comprises a diuretic agent.

### Therapeutic Compositions

An aspect of the present application is directed to a pharmaceutical composition comprising a therapeutically effective amount of an agent comprising or more nitrated fatty acid compounds selected from the group consisting of NO₂-cLA, CP-36, CP-45, CP-46, CP-51, and analogues/derivatives thereof. In some embodiments, NO₂-cLA consists of the 12-isomer form. In some embodiments, NO₂-cLA consists of the 9-isomer form. In some embodiments, NO₂-CLA comprises both the 12-isomer form and the 9-isomer form. In some embodiments, the composition further comprises a pharmaceutically acceptable carrier.

In some embodiments, an agent of the present application is administered orally. Suitable compositions for oral administration include, but are not limited to: tablets, coated tablets, gelcaps, dragées, capsules, powders, granulates and soluble tablets, and liquid forms, for example, suspensions, dispersions or solutions.

In some embodiments, an agent of the present application is administered transdermally.

In some embodiments, an agent of the present application is administered intravenously, intradermally, subcutaneously, or intraperitoneally.

In some embodiments, an agent of the present application is administered as a vaginal suppository.

In some embodiments, the pharmaceutical composition comprises one or more analgesic agents selected from the group consisting of salicylates, aspirin, salicylic acid, methyl salicylate, diflunisal, salsalate, olsalazine, sulfasalazine, para-aminophenol derivatives, acetanilide, acetaminophen, phenacetin, fenamates, mefenamic acid, meclofenamate, sodium meclofenamate, heteroaryl acetic acid derivatives, tolmetin, ketorolac, diclofenac, propionic acid derivatives, ibuprofen, naproxen sodium, naproxen, fenoprofen, ketoprofen, flurbiprofen, oxaprozin; enolic acids, oxicam derivatives, piroxicam, meloxicam, tenoxicam, ampiroxicam, droxicam, pivoxicam, pyrazolon derivatives, phenylbutazone, oxyphenbutazone, antipyrine, aminopyrine, dipyrone, coxibs, celecoxib, rofecoxib, nabumetone, apazone, nimesulide, indomethacin, sulindac, etodolac, diflunisal and isobutylphenyl propionic acid.

In some embodiments, the pharmaceutical composition comprises one or more analgesic agents, individually or in combination, in an amount between 50-2000 mg, 50-1500 mg, 50-1200 mg, 50-1000 mg, 50-800 mg, 50-600 mg, 50-500 mg, 50-400 mg, 50-300 mg, 50-250 mg, 50-200 mg, 50-150 mg, 50-100 mg, 100-2000 mg, 100-1500 mg, 100-1200 mg, 100-1000 mg, 100-800 mg, 100-600 mg, 100-400 mg, 100-250 mg, 250-2000 mg, 250-1500 mg, 250-1200 mg, 250-1000 mg, 250-800 mg, 250-600 mg, 250-400 mg, 400-2000 mg, 400-1500 mg, 400-1200 mg, 400-1000 mg, 400-800 mg, 400-600 mg, 600-2000 mg, 600-1500 mg, 600-1200 mg, 600-1000 mg, 600-800 mg, 800-2000 mg, 800-1500 mg, 800-1200 mg, 800-1000 mg, 1000-2000 mg, 1000-1500 mg, 1000-1200 mg, 1200-2000 mg, 1200-1500 mg or 1500-2000 mg, wherein the composition is formulated for immediate release or for extended release with a release profile in which the one or more analgesic agents are released continuously over a period of 5-24 hours, 5-8, 8-16 hours or 16-24 hours.

In some embodiments, the pharmaceutical composition is formulated for extended release with a release profile in which the active ingredients are released continuously over a period of 5, 6, 7, 8, 10, 12, 14, 16, 18, 20, 22 or 24 hours.

In some embodiments, the pharmaceutical composition is formulated with an immediate release component of active ingredients and an extended release component of active ingredients, wherein the extended release component has a release profile in which the active ingredients are released continuously over a period of 5, 6, 7, 8, 10, 12, 14, 16, 18, 20, 22 or 24 hours. In some embodiments, the immediate release component comprises 5, 10, 15, 20, 25, 30, 35, 40, 45, 50, 55, 60, 65, 70, 75, 80, 85, or 90 percent of the active ingredients.

The pharmaceutical composition may optionally comprise an enteric polymer, and/or at least one excipient, such as a filler, a binder (as described above), a disintegrant, and/or a flow aid or glidant.

Exemplary fillers include but are not limited to, lactose, glucose, fructose, sucrose, dicalcium phosphate, sugar alcohols also known as “sugar polyol” such as sorbitol, manitol, lactitol, xylitol, isomalt, erythritol, and hydrogenated starch hydrolysates (a blend of several sugar alcohols), corn starch, potato starch, sodium carboxymethylcellulose, ethylcellulose and cellulose acetate, enteric polymers, or a mixture thereof.

Exemplary binders, include but are not limited to, water-soluble hydrophilic polymers, such as Povidone (PVP: polyvinyl pyrrolidone), copovidone (a copolymer of polyvinyl pyrrolidone and polyvinyl acetate), low molecular weight HPC (hydroxypropyl cellulose) low molecular weight HPMC (hydroxypropyl methylcellulose), low molecular weight carboxy methyl cellulose, ethylcellulose, gelatin, polyethylene oxide, acacia, dextrin, magnesium aluminum silicate, starch, and polymethacrylates such as Eudragit NE 30D, Eudragit RL, Eudragit RS, Eudragit E, polyvinyl acetate, and enteric polymers, or mixtures thereof.

Exemplary disintegrants include but are not limited to low-substituted carboxymethyl cellulose sodium, crospovidone (cross-linked polyvinyl pyrrolidone), sodium carboxymethyl starch (sodium starch glycolate), cross-linked sodium carboxymethyl cellulose (Croscarmellose), pregelatinized starch (starch 1500), microcrystalline cellulose, water insoluble starch, calcium carboxymethyl cellulose, low substituted hydroxypropyl cellulose, and magnesium or aluminum silicate.

Exemplary glidants include but are not limited to, magnesium, silicon dioxide, talc, starch, titanium dioxide, and the like.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein.

### Example 1—Therapeutic Role of NO₂-FA as a Drug Class in Endometriosis

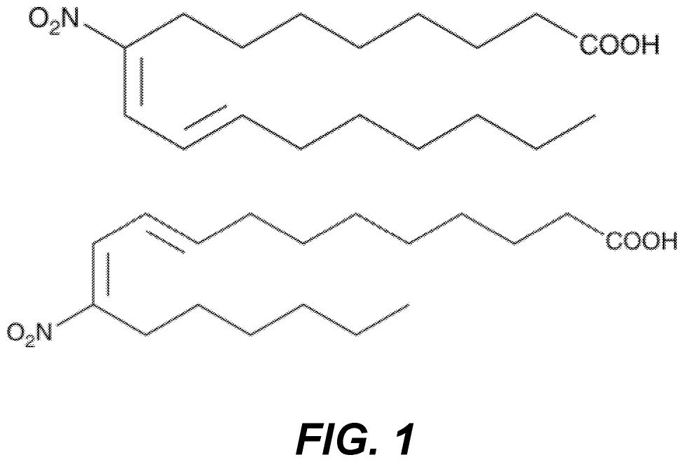

Due to structural and mechanistic homology, Nitro-conjugated linoleic acid (NO₂-cLA) (FIG. 1) exerts many of the same physiological responses as NO₂-OA. NO₂-CLA is the most abundant endogenously produced NO₂-FA and is basally present in the plasma and urine of healthy individuals. Its plasma levels can be further modulated by NO₂-cLA oral supplementation by up to 10-fold, well within cell signaling-relevant concentrations. Dietary oral administration of the precursors, cLA and NO₂— or NO₃—, to healthy volunteers results in plasma free NO₂-cLA concentrations (Cₘₐₓ) reaching up to 25.7 nM and 9.9 nM respectively, comparable to NO₂-OA levels in Phase I trials (mean Cₘₐₓ 0.8 nM (25 mg), 10 nM (150 mg) and 15 nM (300 mg) dose, and supporting the feasibility and significance of NO₂-CLA for clinical applications.

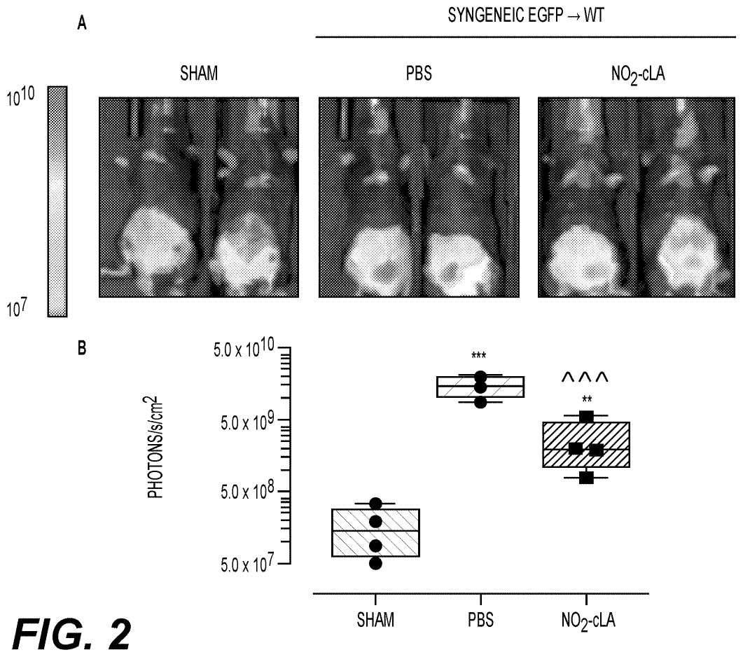

To establish the potential therapeutic role of NO₂-FA as a drug class in endometriosis, this study first tested NO₂-cLA in a well-established syngeneic model of endometriosis. Briefly, 8-weeks old female EGFP transgenic mice (JAX #006567) were treated with PMSG (5 IU/mouse, i.p.) to stimulate endometrial growth and synchronize the estrus cycle. After 3 days, mice were euthanized and each uterine horn was then minced injected into the peritoneal cavity of 8-weeks-old C57BL/6J (wild-type, WT) recipients (2:1, donor: recipient ratio). Mice were randomized to receive NO₂-cLA (2.5 mg/mice, s.c. injection) or vehicle (1% EtOH in PBS) daily for 28 days. In vivo imaging was then used to: 1) establish the syngeneic model by measuring EGFP emission from the peritoneal cavity, demonstrating that the lesions originated from the donor (FIG. 2, panel A); 2) demonstrate that NO₂-CLA administration reduces endometriotic lesions (FIG. 2, panel B), as a proof-of-principle to establish that NO₂-FA efficiently reduce EGFP-derived lesions. The screening approach of NCEs based on NO₂-FA revealed that CP-36 is a more robust and irreversible STING inhibitor rivaling H-151 in potency, a well-known STING inhibitor, yet with a better oral bioavailability and minimal toxicity in vitro and in vivo. Thus, given the intimate link between inflammation and fibrosis in endometriotic lesions, these studies establish that a novel NO₂-cLA-derived STING inhibitor, CP-36, affording the advantage of oral treatment, is a viable candidate for accelerated clinical translation of a novel non-hormonal therapy against endometriosis.

### Example 2—Orally Bioavailable Nitroalkene Compounds as an Endometriosis Therapeutic

A number of nitroalkene compounds with chemical similarity to first generation NO₂-FA were screened for their potential to inhibit STING dependent signaling and inflammation. The study identified CP-36, among others as NO₂-cLA (FIG. 1) lead STING inhibitors.

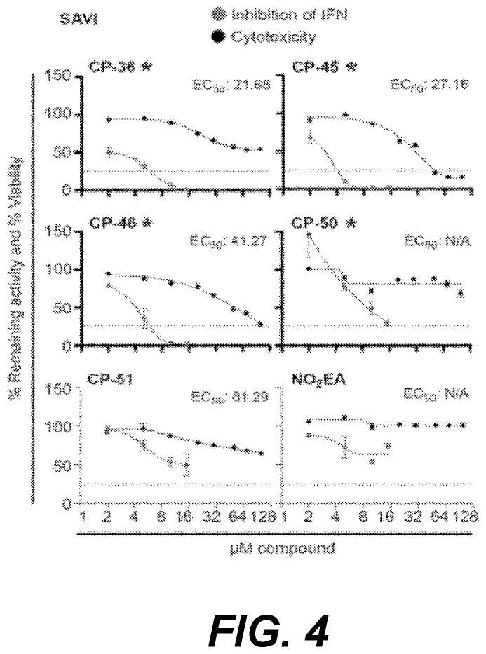

To assess inhibition profiles and pharmacological characteristics of second-generation nitroalkenes, the in vitro therapeutic index was evaluated based on the ability to suppress cGAMP-induced CXCL-10 release and cytotoxicity in THP-1 monocytes. Based on this assay, several conjugated nitroalkene compounds: CP-36, CP-45, CP-46, CP-50, CP-51, and NO₂-EA strongly inhibited the cGAMP-induced CXCL-10 release while showing minimal cytotoxicity evaluated by MTT measurements. The STING inhibitory effects of these selected conjugated nitroalkene compounds were further evaluated in patient-derived SAVI (STING-associated vasculopathy with onset in infancy) fibroblasts bearing the gain-of-function mutation (N154S). CP-36, CP-45, CP-46, and CP-50 dose-dependently reduced significantly IFN expression while showing minimum cytotoxicity (FIG. 4).

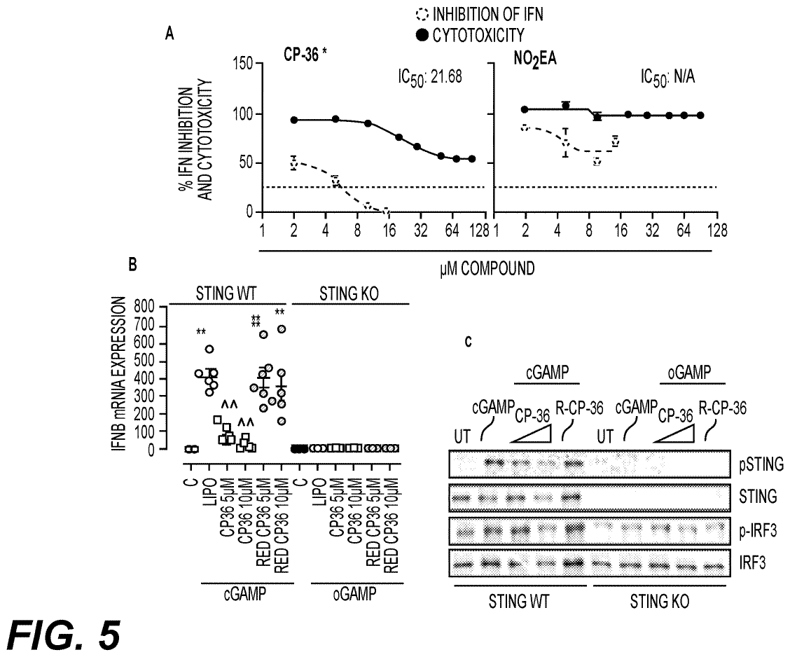

CP-36 was tested for the ability to modulate STING-dependent inflammation in vivo due to its robust STING inhibition while displaying minimal cytotoxicity measured in SAVI patients-derived fibroblasts (FIG. 5, panel A, left side). Others (i.e. NO₂-derived eleostearic acid (NO₂-EA)), an endogenously formed compound recently found in human urine, with structural relationship similarities to CP-36, had negligible STING inhibition capabilities (FIG. 5, panel A, right side). Furthermore, CP-36 dose dependently inhibits cGAMP-induced INFb expression in murine bone-marrow-derived macrophages (BMDMs) and STING activation (phosphorylated STING, pSTING) and downstream signaling (phosphorylated IRF3, pIRF3) (FIG. 5, panel B, and FIG. 5, panel C). Specificity of CP-36 as STING inhibitor was also assessed in BMDMs with equimolar doses of the reduced compound (R-CP36), which lacks electrophilicity and thus reactivity (FIG. 5, panel B, and FIG. 5, panel C).

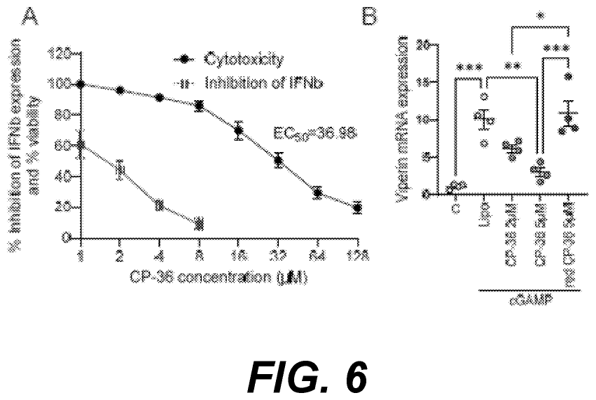

The study tested CP-36 therapeutic index in endometriotic epithelial (12Z) cells by combining analysis of toxicity (MTT assay) and mRNA expression of IFNb (FIG. 6, panel A). Expression of another bona fide STING target, viperin, which has also been identified as critical for inflammation in endometriotic epithelial cells, was dose dependently reduced by CP-36 (FIG. 6, panel B). A major caveat of currently available STING inhibitors, such as nitro-furane derivatives (i.e. H151) is their limited translational potential, as they are poorly orally bioavailable in vivo, albeit being robust STING inhibitors in cellular model systems.

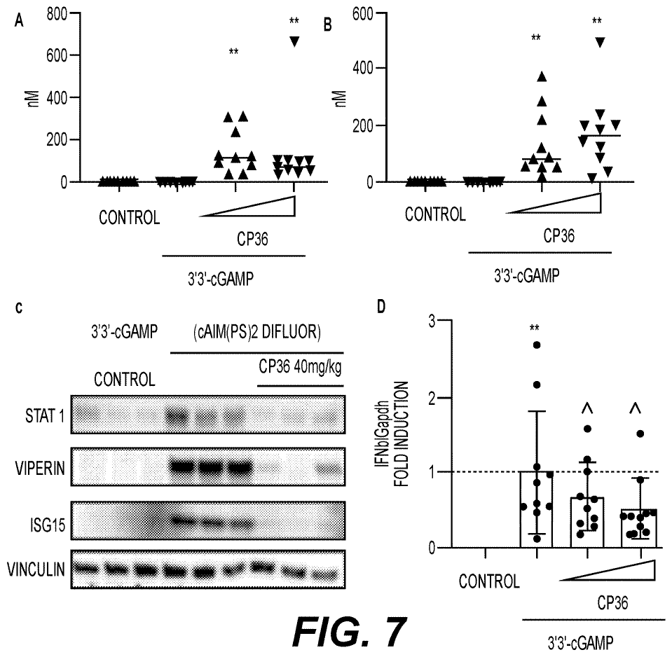

Herein, the study tested bioavailability of CP-36 in the mouse upon oral administration yielding steady state levels at the nanomolar range (FIG. 7, panel A). Mass spectrometry analysis further revealed the presence of dihydro-CP-36 in the plasma (the reduced form required for degradation by fatty acid B-oxidation) (FIG. 7, panel B), demonstrating that CP-36 is readily bioavailable and yield plasma levels well within the therapeutic potential of NO2-FAs Functional analysis on major tissues (liver, heart, lung or spleen) also revealed that CP-36 efficiently inhibits (cAIM(PS)2 Difluor (Rp/Sp), InvivoGen)68-induced type I IFN signaling (i.e., viperin, ISG15), proinflammatory pathways (i.e., STAT1) and IFNB expression in all major organs (FIGS. 7, panel C and FIG. 7, panel D).

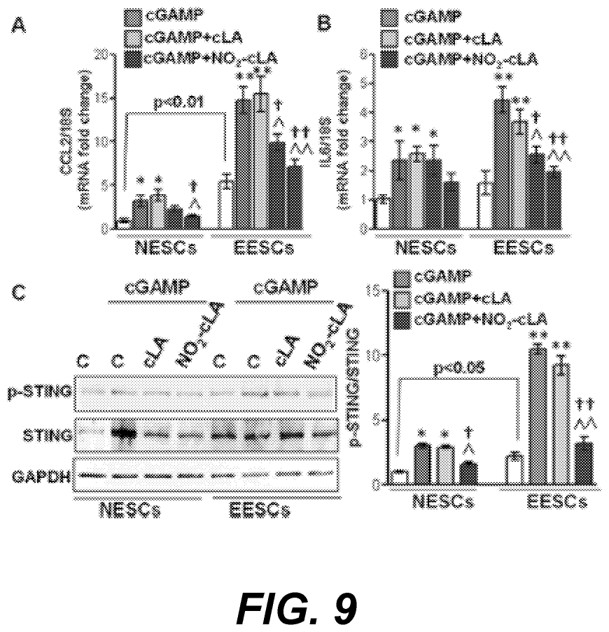

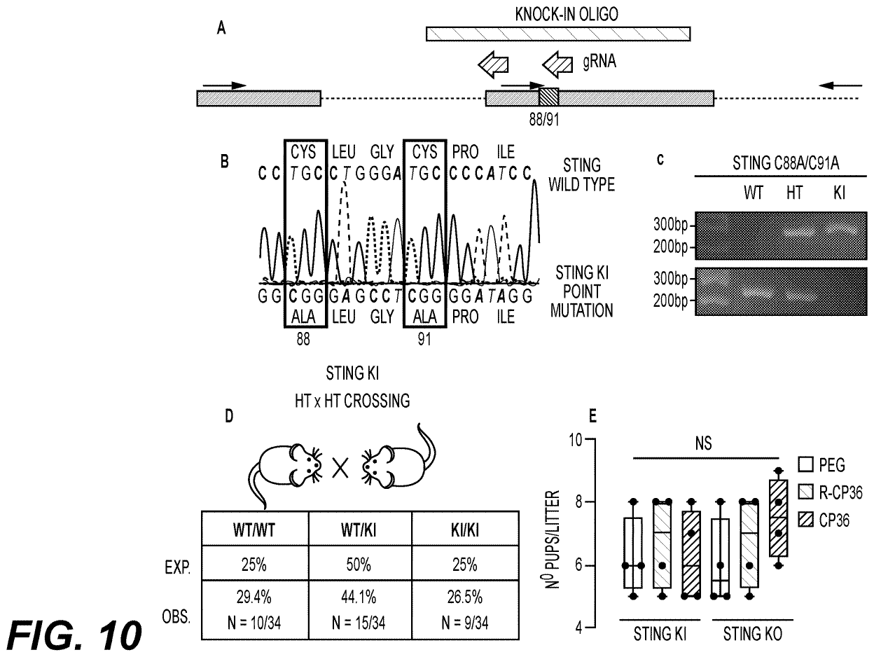

Furthermore, the study found no evidence of reproductive effects of CP-36 (FIG. 10, panel E). Thus, the present study provides a surprising therapeutic approach against endometriosis, due to uncovering novel bioavailable STING inhibitors with no evidence of toxicity in vivo and in endometriotic epithelial cells in vitro. This cell type is of particular importance, as emerging evidence further suggests that aberrant STING expression in epithelial cells is centrally involved in endometriosis development. The data using Tissue Microarray (TMA) from endometriosis patients further reinforce this notion (FIG. 9, panels A-D). This study fills a gap in knowledge of STING roles in endometriosis and its potential for targeting. It is founded on the scientific premises stemming from the present observations of unbridled inflammation in endometriotic cells and lesions, data on anti-inflammatory and anti-fibrotic effects of NO₂-FA and STING inhibitors such as CP-36, their inhibitory effects on endometriotic lesion formation in relevant preclinical syngeneic models, repression of inflammatory gene expression, and preliminary data demonstrating CP-36 oral bioavailability and STING inhibition in endometriotic epithelial cells.

This study translates new knowledge regarding the identification and actions of the New Chemical Entity (NCE) CP-36, as a novel STING inhibitor, into a feasible oral therapeutic approach against endometriosis, while simultaneously contributing to incisive mechanistic insight on the role of STING as a key pro-inflammatory pathway for this devastating disease, currently lacking effective non-hormonal drug treatments.

### Example 3—Upregulation of STING in Endometriosis

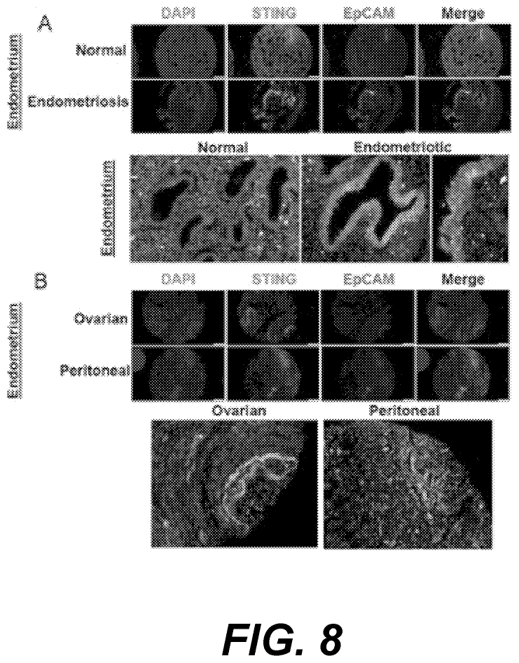

As STING is overexpressed in human endometriosis, the study used a tissue microarray (TMA) containing samples from control endometrium and endometriosis to assess STING localization. STING immunofluorescence (green) was uniformly co-localized with epithelial cell marker EpCAM (red) in the glands and stroma of endometrium. STING and EpCAM were intense but just in the endometriotic uterine glands and stroma compared to normal endometrium. However, the intensity of STING expression in endometriotic endometrium glands was higher than in the normal endometrium (FIG. 7, panel A). Further analysis of endometriotic ovarian and peritoneal tissues showed both STING and EpCAM intensely colocalized in ovarian lesions and peritoneal tissues with weaker STING immunofluorescence in the stromal layer. (FIG. 8, panel B). To determine the therapeutic potential of NO₂-FAs against endometriosis, STING activation was evaluated in eutopic endometriotic stromal cells (EESCs) derived from endometriosis patients and from normal endometrial stromal cells (NESCs) derived from biopsies from normal patients. EESCs displayed an overall enhanced inflammatory phenotype compared to normal NESCs as indicated by increased expression of proinflammatory cytokines CCL2 and IL-6 (FIG. 9, panel A and FIG. 9, panel B). These findings of overall inflammatory phenotype are consistent with previous reports by our team and others. STING activation upon treatment with the selective activator, cyclic GMP-AMP (cGAMP) resulted in significantly increased of CCL2 and IL6 mRNA expression in EESCs compared to NESCs (FIG. 9, panel C). STING activity was also more pronounced in EESCs compared to NESCs, as demonstrated by western blot analysis of phosphorylated STING (p-STING) vs. total STING (FIG. 9, panel C), further reinforcing the notion that STING activation is functional in EESCs and promotes pathology. Of relevance, treatment with NO₂-cLA, but not the non-nitrated counterpart (cLA), inhibited cGAMP-induced proinflammatory CCL2, IL6 mRNA expression in EESCs in a dose dependent manner (FIG. 9, panel A and FIG. 9, panel B) while STING activation was significantly reduced by NO₂-cLA, whereas cLA has no effect (FIG. 9, panel C).

### Example 4—STING KI Mice with Specific Cys to Ala Substitution

CRISPR/cas9 was used to generate the double mutant STING C88A/C91A knock-in mouse (STING KI) that will be used in this proposal to address specificity of CP-36 binding to STING in vitro and in vivo and for in vivo functional studies (FIG. 10, panel A and FIG. 10, panel E). Microinjection of the Cas9 mRNA, sgRNA and C88A/C91A KI donor oligo and embryo transfer were performed at the University of Michigan Transgenic Core. Off-target effects were addressed via gRNA analysis with CRISPOR and through backcrossing the F0 carrying the KI mutation and subsequent generations with C57BL/6 mice (>10 rounds). Animals are available at MSM. Overall, STING KI strain, like STING KO mice (as reported by JAX), has a normal reproductive behavior with the expected Mendelian ratios when mating HTxHT for the KI mutation (FIG. 10, panel C). Also, oral administration of CP-36 or Red-CP-36 to confirmed (presence of plug) pregnant females yielded similar reproductive rate (FIG. 10, panel D) with no differences of endometrial receptivity markers (i.e. GM-CSF, LIF, VEGF) assessed by Milliplex assays in vaginal smears of STING KI, STING KO or upon treatment with CP-36 (data not shown).

These in vitro preliminary data in combination with the demonstration that oral CP-36 is readily bioavailable and efficiently inhibits STING signaling in vivo (FIG. 6, panel A) support that STING is a target of direct nitroalkylation and that second generation CP-36 inhibits the STING>TBK1> IRF3 inflammatory pathway in endometriosis. Accordingly, the study pursues a series of in vitro and ex vivo experiments to assess: 1) that STING is a functional target of CP-36 in eutopic endometriotic cells; 2) in vitro markers of inflammation, fibrosis, and angiogenesis in response to CP-36 using both eutopic and ectopic endometriotic cells from WT, STING KI and STING KO mice; 3) Towards a translational approach, STING-mediated inflammation will be evaluated in response to CP-36 using organoid system from EESCs isolated from women with endometriosis and NESCs without endometriosis procured through archived samples.

### Example 5—Determination of STING Nitroalkylation and Functional Inhibition of STING Signaling by CP-36

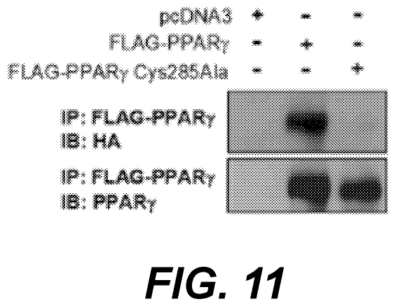

Bio-orthogonal labeling (i.e., click chemistry) has become an important tool for scientists to minimally modify molecules to then, using a highly specific ex vivo labeling strategy, identify them. The study implemented bio-orthogonal labeling assisted unbiased proteomics to evaluate CP-36 targets, as exemplified in FIG. 11. PPARγ is a target for NO₂-OA at Cys285 in the binding pocket. To address the specificity of this technology, we overexpressed FLAG-tagged-PPARγ WT vs. FLAG-PPARγ C285A mutant in HEK293T cells and treated with Az-NO₂-OA (labeled on the @-carbon). Az-NO₂-OA reacted covalently with Cys285 PPARγ-FLAG (WT), as detected by immunoprecipitation with anti-FLAG antibody and “clicking” the Az-NO₂-OA with alkyne-HA, while specificity was ascertained by loss of signal in the C285A mutant (FIG. 11). This approach can be utilized to identify protein targets responsible for the acute effects and signaling events of CP-36 in the endometrium. For the specific purpose of this proposal, functional STING targeting by CP-36 (nitroalkylation) is pursued in eutopic endometrial epithelial and stromal cells and ectopic (derived from the peritoneal lesions) in C57BL/6J mice. STING KI female mice are used to assess the specificity of CP-36 adduction in these specific residues within the STING protein, as a validation methodology. WT and STING KI female mice are synchronized and standardize the phase of the estrous cycle, as described through daily i.p. injection of E2 (100 ng) for 3 consecutive days. The estrous cycle is assessed by the ratio of desquamated cells in vaginal lavages. To demonstrate CP-36 binding to STING, bio-orthogonal labeling is performed subsequently under two experimental settings: 1) in vitro, using freshly isolated endometrial epithelial and stromal cells99 from the experimental mouse models, and treated for 3 h with Az-NO₂-CP-36 or inactive (reduced) Az-R-CP-36100 in a dose-dependent manner (1, 2, 5, 10 μM); parallel experiments will be pursued to assess inhibition of STING palmitoylation. For these experiments cells are treated with azpalmitate prior treatment with CP-36 or R-CP-36. 2) In utero bio-orthogonal labeling: Syngeneic endometriosis is performed in WT and STING KI mice using WT uterine fragments as donor or sham controls as described above. Endometriosis is allowed to progress for 2 weeks. Az-CP-36 or Az-R-CP36 (10 mg/kg, i.v. for distribution to the tissues of interest) is injected into WT and STING KI mice (synchronized at the estrus phase, n=5 per group); 2 h post injection, eutopic endometrium (uteri) and ectopic endometrium (peritoneal lesions) are collected and flash frozen. After thawing, tissue homogenates are clicked with Click-iT Protein Enrichment Kit (Invitrogen) and analyzed as in FIG. 10, panel C, with anti-STING antibodies.

### Example 6—Inhibition of STING by CP-36 Inhibits Endometriosis

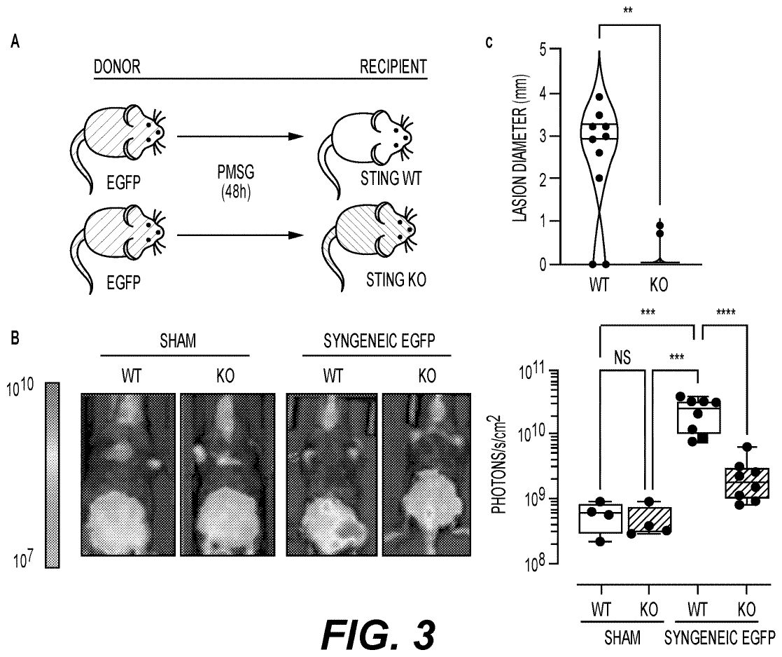

The study defines STING inhibition by CP-36 as a necessary condition for anti-inflammatory mechanisms in endometrial epithelial and stromal cells following an equivalent experimental approach as described above. Epithelial and stromal cells are isolated from the uterus of mice subjected to syngeneic endometriosis versus sham from WT and STING KI female following well-established procedures. To assess purity of the isolation, vimentin staining is used as a stromal cell marker and cytokeratin for an epithelial cell marker. Only cells achieving 90% of vimentin or for cytokeratin is used. In comparative analyses to study whether inhibition of STING-signaling by CP-36 attenuates inflammatory responses, cells isolated from WT and STING KI female mice are stimulated with the STING agonist cGAMP (FIG. 3) (4 ng/mL) and treated with CP-36 in a time-(1-24 h) and dose-dependent (0.5-5 μM) manner. Equimolar concentrations of the reduced R-CP-36 are used as control. Outcomes are 1) mRNA (RTqPCR) and protein expression (western blot) of pro-inflammatory cytokines involved in endometriotic pathogenesis: (INFB, CCL2/MCP-1, RANTES, ILIB, IL6, IL-10) and fibrosis: (Col1, Col3, Fibronectin, CTGF); adhesion molecules (VCAM-1 and ICAM-1), invasive factors (MMP-2, MMP-9, TIMP-1 and TIMP-2), angiogenic factors (VEGF-A, VEGFR1 and VEGFR2), proliferation markers (CDK1/4, Ki67, p21, p27) and apoptotic factors (Caspase-3, Caspase-9, Bax and BcL-2). 2) STING signaling in response to CP-36 is analyzed at the level of expression (STING) and activation (p-STING) by western blot; 3) STING inhibition by CP-36 is further assessed by analyzing downstream signaling, including pTBK1 and pIRF3, by western blot normalized by total TBK1 or IRF3, respectively. Co-immuno-precipitation studies are pursued to address the interaction of STING with TBK1 and IRF3 signaling and STING downstream signaling (IRF3, IFIT1), in response to CP36 following cGAMP stimulation. 4) Inflammatory cytokines chemokines obtained from the cell supernatant are analyzed by Mouse Cytokine/Chemokine Assays (Millipore) coupled with xMAP Intelliflex system. The study follows a focused approach to identify STING Cys88/Cys91 as target of nitroalkylation in cells and organoids in vitro and in the uterus in vivo in the context of syngeneic endometriosis, using unique STING knock-in strategies and models.

Bio-orthogonal labeling of CP36 followed by click chemistry will identify Cys88 and/or Cys91 of STING as targets of CP-36 adduction. Specificity of the reaction is demonstrated in cells obtained from STING KI mice (loss of adduction) as well as in the uteri in vivo. The inclusion of reduced Az-R-CP-36, which lacks electrophilic reactivity, is an essential control. The inflammatory component of endometriosis is a requisite for increased STING activation as demonstrated in FIG. 7, panels A-D and FIG. 8, panels A-B. Thus, bio-orthogonal labeling of STING with Az-CP-36, even administered by i.v. injection, may result in low yields in the normal uteri (sham) and the click reaction may fall below the limits of detection. Yet, in the context of syngeneic endometriosis aberrantly expressed STING will likely yield sufficient sensitivity for CP-36/STING adduction. Alternatively, uterine inflammation is induced via intrauterine injection of LPS 3 h prior to the Az-CP-36 (or Az-R-CP-36) i.v. treatment, as previously described or specific STING activation by cGAMP. STING phosphorylation is monitored as surrogate for STING activation (FIG. 5, panels A-C; FIG. 7, panels A-D; and FIG. 8, panels A-B). The functional consequence of STING inhibition by CP-36 is evidenced by inactivation of the STING signaling via TBK1 and IRF3 phosphorylation, cytokine production, etc. Inhibition of TBK1 and IRF3 phosphorylation by CP-36 is expected in WT but not in STING KI either assessed in epithelial and stromal cells or in the syngeneic endometriosis model, thus defining a functional role of STING Cys88/Cys91 as targets of CP-36. No adduction or alteration of TBK1 and IRF3 phosphorylation is expected by R-CP-36. Although obviated in attention to “3 Rs principle,” NO₂-CLA & cLA could be introduced as reference in key experiments for in vivo and in vitro comparison of CP-36 & R-CP-36. Key findings may also be assessed in organoid systems from WT and STING KI; or derived from human iPSCs STING WT or Cis88A Cis91A mutated via CRISPR/cas9 (LoS from Emory iPSC Core). Multiple interdependent post-translational modifications (PTMs) allow for combinatorial repertoires of interactions. In STING, some of these modifications, i.e. phosphorylation, palmitoylation and nitroalkylation, might present crosstalk for dynamic control of cellular signaling under various pathophysiological conditions. The study keeps the focus here on STING palmitoylation and its competitive and irreversible inhibition by CP-36, yet has the capability to deepen the mechanistic studies to address CP-36 effects of on those additional NO₂-FA targets of PTM.

Because of the nitroalkene nature of CP-36, alternative pathways to STING are considered. For instance, NO₂-FAs have been shown to have anti-inflammatory effects via direct adduction of the p65 component of NF-κB. NF—KB or the STAT pathway (FIG. 6, panels A-B) is inhibited by CP-36, as demonstrated in several preclinical studies. STING and NF-κB signaling may act in concert to mount an inflammatory response. Cells derived from STING KI mice, compared to WT, can allow the determination of the relative contribution of NF-κB to the effects of CP-36 since STING nitroalkylation is not expected in endometriotic cells from STING KI mice. Analysis of TBK1 activation (i.e., pTBK1) will further inform NF-κB contribution as both STING and non-canonical NF-κB pathways converge on TBK1 activation during inflammatory responses, including in endometriosis. Additionally, studies identified Nrf2 and PPARγ activation as key signaling pathways regulated by NO₂-FA, which are also known to play important roles in endometriosis. All analytical tools, including biochemical characterization and experimental models (i.e. NF-κB-luciferase transgenic mouse), PPARγ activity or Nrf2 are available to evaluate their contribution to inhibition of endometrial inflammation by CP-36. Example 7-Oral delivery of CP-36 reduces endometriosis

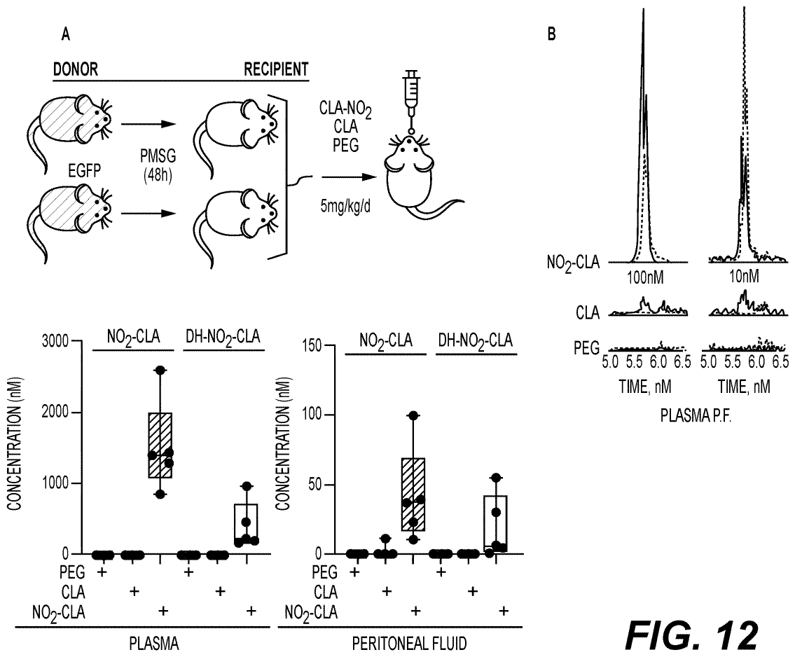

To establish the potential therapeutic role of NO₂-FAs against endometriosis, proof-of-principle studies with subcutaneous delivery of first generation NO₂-FAs were pursued, demonstrating that NO₂-cLA administration efficiently reduces EGFP-derived endometriotic lesions (FIG. 2). Oral bioavailability is a key characteristic in drug development as it significantly increases the reach of the therapeutic approach and avoids invasive delivery methods such as s.c. delivery. Thus, the study tested whether administering NO₂-cLA orally seemingly reduces syngeneic endometriosis (FIG. 12, panels A-E) following well-established protocols developed in the study, including the use of PEG as vehicle to stabilize NO₂-cLA and using equal doses of cLA as non-nitrated control. First, we assessed if NO₂-cLA was absorbed and systemically distributed. As expected, free NO₂-cLA and its beta oxidation metabolite were readily detected in plasma (FIG. 12, panel A) confirming widespread biodistribution at similar levels than those detected with CP-36, a more potent STING inhibitor (FIG. 6, panels A-B). Most relevant for peritoneal endometriosis, NO₂-cLA was also detectable at therapeutic doses at the peritoneal fluid in our syngeneic model of endometriosis, suggesting that oral nitroalkene delivery reaches relevant target tissues (FIG. 12, panel B). Of importance, our new preliminary data revealed that oral NO₂-cLA persistently protects against endometriosis as assessed by non-invasive imaging and endpoint lesion size determination, while cLA has no effect (FIG. 12, panels C-E).

### Example 8—STING KO Mice are Protected from Endometriosis and Display Reduced Pain Markers

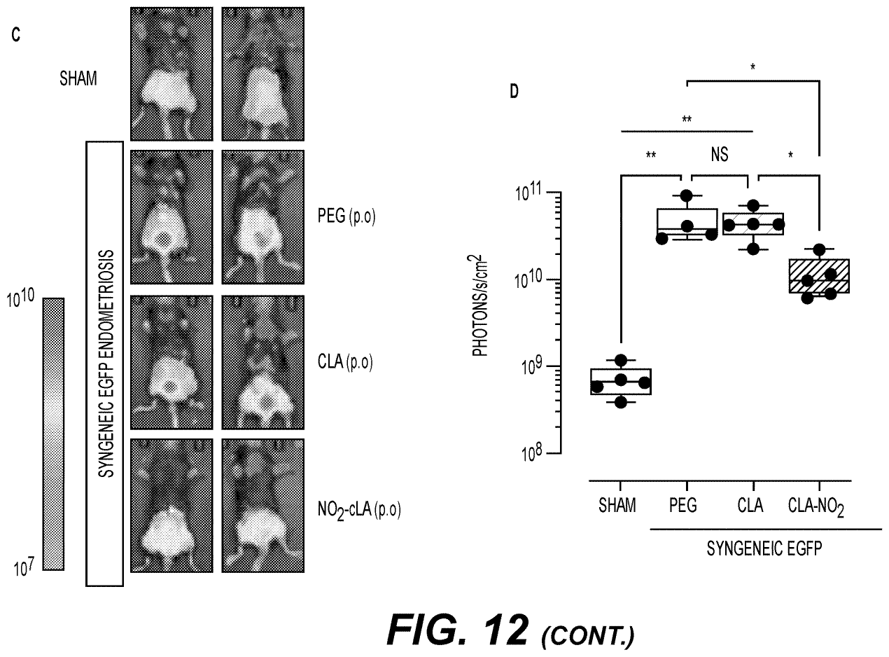

To assess STING contribution to endometriosis, parallel experiments were pursued in which EGFP-derived uterine fragments were injected into the peritoneum of STING KO and WT mice. In vivo imaging demonstrated that WT mice developed significant endometriotic lesions, whereas EGFP emission was significantly reduced in STING KO mice (FIG. 3, panels A-B). A parallel experiment was further pursued at an earlier time-point (1 week post syngeneic transplantation) for plasma and peritoneal fluid cytokine profiling. As expected, levels of IFNb were reduced in STING KO mice in our syngeneic model (FIG. 13, panels A). Importantly, fractalkine, a bona fide marker of pain in endometriosis, was significantly reduced in both plasma and peritoneal fluid of STING KO mice (FIG. 13, panels B).

### Example 9—Loss of STING Function Reduces Endometriosis Lesion Formation

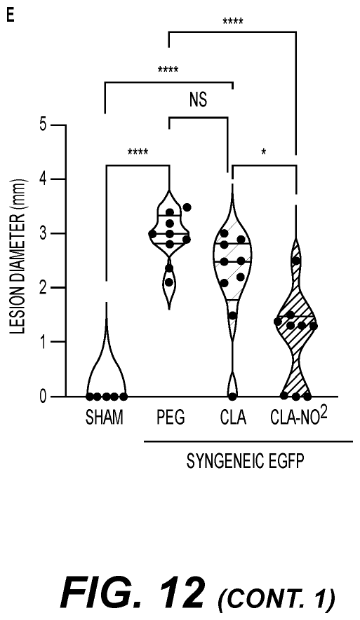

Because STING was differentially expressed in the endometrium in humans, a reverse approach of the syngeneic model was conducted to determine the effect of STING KO mice as donors (FIG. 3A). Uterine fragments from PMSG-treated STING KO and WT mice were transplanted into the peritoneal cavity of C57BL/6J recipient mice. Endometriosis was allowed to progress for 28 days followed by lesion size determination. STING KO mice had fewer lesions that were significantly smaller in diameter compared to WT (FIG. 14, panels A-C). Proinflammatory cytokine expression in the host did not differ, suggesting that STING function in the uterine fragments also contributes to endometriosis regardless of the inflammatory status of the recipient. As shown above, oral CP-36 is readily bioavailable and efficiently inhibits STING signaling in endometriotic epithelial cells in vitro (FIG. 6, panels A-B). Accordingly, the study systematically assesses endometriotic lesion formation upon oral delivery of CP-36 and its dependence on STING inhibition in endometriosis models in vivo. The immunocompetent syngeneic EGFP endometrial stromal injection model (FIG. 2, panels A-B; FIG. 3, panels A-C), provides the benefit of increased sensitivity in endometriotic lesions identification and quantification of lesion size and growth rates. This model is used to a) monitor STING contribution to early endometriotic implantation, and b) to assess STING-dependent CP-36 protection against endometriotic lesion progression (Examples 9, 10) and regression (Example 11). Overall these studies 1) establish whether inhibition of STING by CP-36 is effective on endometriosis inhibition and 2) assess endometriosis-associated pain. STING KI mice (FIG. 10, panels A-E) serve as a unique experimental model to provide key in vivo proof-of-concept to define CP-36 as a potential drug therapy for endometriosis specifically targeting STING. Unlike STING KO, the STING KI approach achieves comparable STING activation as WT counterparts in response to inflammatory stimuli yet remaining insensitive to CP-36 regulation. Based on preliminary data, the study shows oral delivery of CP-36 reduces endometriosis via inhibition of inflammation and fibrosis and associated pain in a STING-dependent manner. The studies 1) characterize STING-dependent inhibition of endometriotic inflammation and fibrosis by CP-36 vs R-CP-36, and 2) define oral administration of CP-36 as a feasible therapy against endometriosis by promoting regression of established endometriosis lesions and associated visceral pain.

### Example 10—Syngeneic Endometriosis Lesions from STING KI Mice

Initial studies pursue a comparative analysis of endometriosis potential of WT and STING KI mice when used as donors. STING KO are used as loss-of-STING function controls. To this aim, WT, STING KI and STING KO mice are crossed to EGFP mice to obtain the following strains: 1) EGFP/WT, 2) EGFP/STING KI, and 3) EGFP/STING KO mice (FIG. 12, panels A-E). Female mice from each EGFP+ strain are then treated with PMSG for 48 h and each uterine horn used as donor for the syngeneic injection into C57BL/6J female mice (FIG. 14, panels A-C). An unbiased assessment of endometriotic lesion formation is performed by in vivo imaging, in a time-dependent manner (1, 2, 3, 4 weeks post endometriosis induction, n=8/time point). An early time-point group (Day 2) is included for analysis of EGFP+tissue engraftment into the peritoneum.

Analysis of peritoneal endometriosis: recipient mice are euthanized by CO₂ overdose at the indicated times after syngeneic endometriosis. The number, location, and sizes of ectopic lesions are assessed under a dissecting microscope using a caliper as described. Histological evaluation and analysis of EGFP intensity will also be pursued, as our team reported before.

Analysis of vascularization: Endothelial cells in endometriotic lesions, the surrounding peritoneum and eutopic endometrium are stained for CD31 by immunohistochemistry. The following scores are applied to measure vascularization: a) CD31 (0, no CD31 immunoreactivity per field; 1, ≤10 CD31+ cells; 2, between 10 and 20 CD31+ cells, 3, ≥20 CD31+ cells); and b) vascularity (0, no blood vessels; 1, 1-5 vessels; 2, 5-10 vessels; 3, ≥10 vessels).

Analysis of immune cells in the peritoneum: Peritoneal exudates are obtained via PBS lavage from each group. An early neutrophil (PMNs) infiltration is expected in the peritoneal cavity upon establishment of syngeneic transplantation, followed by monocyte and macrophages infiltration. Thus, analysis is focused on PMNs, monocytes and macrophages in peritoneal endometriosis using flow cytometry, based on the following markers: CD11b+, (Ly6G)+ for neutrophils (PMNs); CD11b+, (Ly6G)−, Ly6Chi for pro-inflammatory monocytes, and CD11b+, F4/80+ for macrophages. Staining specificity is verified with isotype-matched antibody controls and compensation performed with single-stained and Fluorescence-Minus-One samples. Acquisition is performed using a BD FACS Aria II available at the MSM Core, and data analyzed by FlowJo. The following analysis is pursued: Tissues are processed for histopathology and immunolocalization. Lesions are then fixed in 4% (w/v) paraformaldehyde and embedded in paraffin for histopathology, immunohistochemistry (IHC) and immunofluorescence analyses. The endometriotic epithelium and stroma are visualized by H&E staining. Fibrosis is evaluated by Masson-Trichrome staining and immunohistochemistry against MMP-9 and TIMP-1. Proliferation and apoptosis in endometriotic implants and eutopic endometrium are monitored by immunolocalization of PCNA, Ki67, caspase 3 and TUNEL staining. Tissue sections are stained with antibodies against monocytes/macrophages (MOMA-2), vascular cell adhesion molecule (VCAM), intercellular adhesion molecule (ICAM), neutrophils (Ly6G), monocytes (Ly6c), and macrophages (F4/80); and assessment of angiogenesis. STING activation and protein levels are determined by immunohistochemistry using p-STING and total STING antibodies, and TBK1 and IRF3.

### Example 11—Endometriotic Growth Inhibition by CP-36

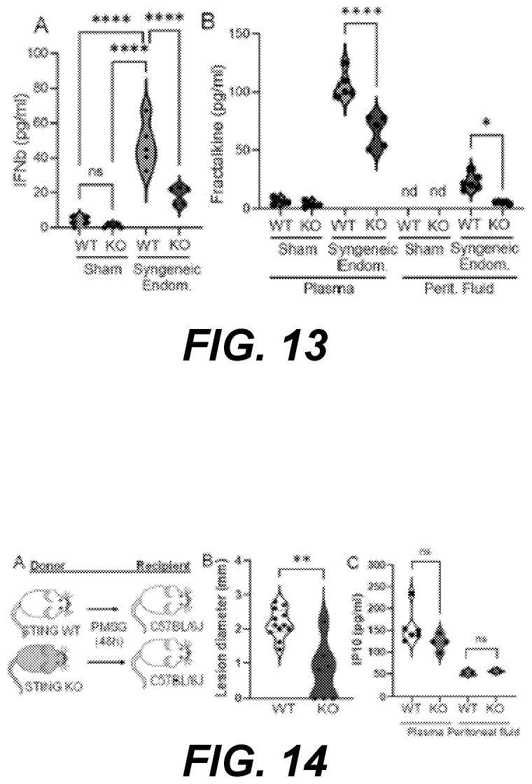

STING KI/EGFP and WT/EGFP mice are used as donors to comparatively analyze and establish the therapeutic potential of CP-36 on the onset and progression of the inflammatory and angiogenic response in endometriotic lesions in the syngeneic EGFP injection model, implemented as in (FIG. 12, panels A-E). Sham implantation consists of i.p. injection of equal volumes of PBS (FIG. 12, panels A-E). Mice are randomized into the following groups: 1) PBS/PEG (2:1 v/v) serving as vehicle control, 2) Reduced CP36 (R-CP-36) serving as non-electrophilic CP-36 control (40 mg/kg) and 3) CP-36 as the therapeutic group (40 mg/kg). Drugs are administered by oral gavage daily as in FIG. 7, panels A-D and FIG. 12, panels A-E. At 2 weeks, urine and feces is collected for quantitative analysis of CP-36 and/or derived metabolites as in FIG. 12, panels A-E. At end point, 4 weeks, mice are euthanized for macroscopic and microscopic assessment of the endometriotic lesions, which are identified in situ via EGFP fluorescence. Endometriotic implant size is measured using a caliper as in Example 9. The implants, uterine horns, ovary, and peritoneal samples are collected. End-point determinations also include plasma collection for MS/MS analysis of CP-36 levels, saturation products and β-oxidation metabolites (FIG. 7, panels A-D) and overall inflammation. Immune cell infiltration is analyzed in peritoneal exudates and in non-fixed lesions as in Example 9. Inflammatory cytokines and chemokines from the peritoneal lavage are also be analyzed by Milliplex assays. The effects of CP-36 on lesions histopathology, fibrosis, angiogenesis, proliferation and apoptosis markers, immune cell adhesion and activation of STING, TBK1 and IRF3 are performed as in Example 9. Comparisons are set among vehicle, R-CP-36 and CP-36 (n=8) and statistical analysis is performed blinded to treatment. Pain assessment (FIG. 15) is performed as described below.

### Example 12—STING Inhibition by CP-36 as a Therapeutic Target for Endometriosis

The therapeutic benefits of NO₂-FAs are primarily related to anti-inflammatory and anti-fibrotic mechanisms. In most cases, however, administration of NO₂-FA was initiated at the onset of disease progression. More recently, the present inventors demonstrated that NO₂-FA can be therapeutically administered once the disease under study has been diagnosed, thus mimicking a more realistic clinical scenario. This is particularly relevant for endometriosis, which can remain undiagnosed for years. Example 10 assessed CP-36 on endometriotic progression from the onset. Herein, treatment is randomized at more advanced stages of endometriosis, once endometriotic lesions with fibrosis (week 4) are established (FIG. 15). To this aim, the syngeneic model is used in which uterine fragments from EGFP/WT, EGFP/STING KI and EGFP/STING KO mice serve as donors onto syngeneic WT recipients. Endometriosis will be followed by Xenogen IVIS Imaging and allowed to progress for 4 weeks, when a subset of the recipient mice will be sacrificed to assess basal endometriotic lesion size and pathology. A second subset will then be randomized for oral treatment with CP-36 or red R-CP-36 as in Example 10. End-point analyses at week 8 will be performed as described in Examples 9 and 10.

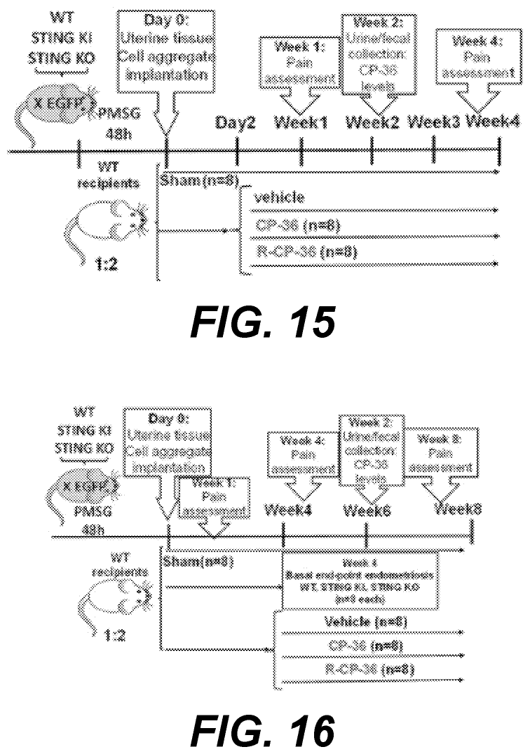

Pain assessments in Examples 9 and 10: Behavioral assessment is made prior to peritoneal injection (Examples 10 and 11) or at the indicated time points in each case (FIGS. 15, 16); and 2 days prior to endpoint. Tests include:
1) Primary viscero-specific pain models: including abdominal discomfort, by recording the number of times the mouse licks, grooms, or barbers the abdominal region as an indicator of local abdominal discomfort i.e. primary/local pain.
2) Secondary (referred) pain behavior models 1) mechanical nociception using von Frey fibers to assess the mechanical threshold required to elicit paw withdrawal (50% paw withdrawal threshold) 2) thermal nociception (Hargreaves method) to determine latency to paw withdrawal using a Plantar Test Analgesia Meter (IITC Life Science). Behavioral tests are run from least stressful to most invasive (licking, Von Frey, Hargreaves) by researchers (interobserver reliability, r>0.90) blinded to treatments.

The study defines the feasibility of CP-36 as an oral therapy against endometriosis by inhibiting STING-mediated inflammation and fibrosis using a well-established syngeneic model. Example 9 provides a comprehensive analysis of the contribution of STING to endometriotic cell engraftment, and endometriotic lesion formation in a time-dependent manner. In Example 9, the number and size of the lesions derived from STING KO mice is reduced compared to WT or STING KI mice. Differences in WT and STING KI mice is expected if the KI mutation affects basal levels of STING activity.

In addition, analysis of peritoneal immune cells will allow us to assess the contribution of the recipient (and potentially the donor) to the inflammatory microenvironment. A time dependent analysis of vascularization will allow to infer differences between levels of inflammatory and angiogenic factors released by implants from donor WT or STING KI. CP-36 administration (vs R-CP-36) will reduce PMNs, pro-inflammatory monocyte infiltration and endometriotic growth upon endometriosis progression (Example 10), and once endometriosis is established from WT but not STING KI donors. As in other models, CP-36 treatment results in lesion size regression and promotes inflammation resolution with reduced pro-inflammatory cytokine production. It is possible that STING KI mice, in which the mutation prevents STING targeting, shows partial inhibition of inflammation by CP-36 as NF-κB inhibition/PPARγ activation by NO₂-FAs may also contribute to the reduced endometriotic lesion formation. This can be addressed directly by monitoring NF-κB pathway simultaneously. CP-36 combined effects on STING, TLR/NF-κB and PPARγ would indeed enhance the significance of the studies. To deepen and expand the mechanistic studies, lesions from each Example are subjected to bulk and scRNAseq. Oral CP-36 delivered to pregnant mice did not result in any apparent side-effect (FIG. 10, panels A-E). However, vigilant monitoring is maintained for adverse effects as it is relevant for the long-term goal at the clinical phase, to optimally maintain a favorable risk-benefit balance for endometriosis patients. Pain may be due to nociceptive, inflammatory, or neuropathic mechanisms, and different types of pain have been associated with endometriosis. Yet, the relationships of endometriosis severity and the presence of pain is poorly understood due to the intrinsic heterogeneity of endometriosis. Oxidative insult and prostaglandins have been shown amongst the drivers of endometriosis pain. It is expected that CP-36-mediated inhibition of endometriotic lesions and inflammation translates into a reduction of local pain as CP-36 inhibits nociceptive signals of importance in endometriosis. Yet, it is possible that secondary pain might be enhanced in STING KO under basal conditions, as STING was shown to regulate nociception in sensory neurons. This study provides a systematic analysis of pain associated with endometriosis and the contribution of STING to further support CP-36 as a novel therapy against endometriosis. Our work focuses on targeting STING from the perspective of inhibition of endometriosis progression and regression, using STING genetic models as donors throughout. Systemic inhibition of STING prevents the inflammatory response in the recipient as well. This can be addressed in parallel experiments by using WT, STING KO and STING KI as recipients with EGFP WT donors in experiments as those in Examples 9-11.

While various embodiments have been described herein, it should be understood that such disclosures have been presented by way of example only and are not limiting. Thus, the breadth and scope of the subject compositions and methods should not be limited by any of the above-described exemplary embodiments but should be defined only in accordance with the following claims and their equivalents.

The above description is for the purpose of teaching the person of ordinary skill in the art how to practice the present application, and it is not intended to detail all those obvious modifications and variations of it which will become apparent to the skilled worker upon reading the description. It is intended, however, that all such obvious modifications and variations be included within the scope of the present application, which is defined by the following claims. The claims are intended to cover the components and steps in any sequence which is effective to meet the objectives there intended, unless the context specifically indicates the contrary.

What is claimed is:

1. A method for the treatment of a subject having an abnormal condition of the reproductive system, the method comprising:
administering to the subject an effective amount of a nitrated fatty acid compound selected from the group consisting of NO₂-cLA, CP-36, CP-45, CP-46, CP-51, and analogues/derivatives thereof.

2. The method of claim 1, wherein the nitrated fatty acid compound is CP-36.

3. The method of claim 1, further comprising administering to the subject one or more analgesic agents.

4. The method of claim 3, wherein the one or more analgesic agents are selected from the group consisting of aspirin, ibuprofen, naproxen, naproxen sodium, indomethacin, nabumetone, and acetaminophen.

5. The method of claim 1, further comprising administering to the subject one or more hormonal contraceptives.

6. The method of claim 1, further comprising administering to the subject one or more progestins.

7. The method of claim 6, wherein the one or more progestins are selected from the group consisting of norethindrone, norethindrone acetate, ethynodiol diacetate, levonorgestrel, desogestrel, gestodene, norgestimate, drospirenone, and medroxyprogesterone acetate.

8. The method of claim 1, further comprising administering to the subject one or more GnRH agonists.

9. The method of claim 8, wherein the one or more GnRH agonists are selected from the group consisting of leuprorelin or goserelin.

10. The method of claim 1, further comprising administering to the subject one or more GnRH antagonists.

11. The method of claim 10, wherein the one or more GnRH antagonists are selected from the group consisting of elagolix, relugolix, and linzagolix.

12. The method of claim 1, further comprising administering to the subject one or more aromatase inhibitors.

13. The method of claim 12, wherein the one or more aromatase inhibitors are selected from the group consisting of anastrozole, letrozole, and exemestane.

14. The method of claim 1, further comprising administering to the subject a diuretic agent.

15. A method for the treatment of infertility in a subject, the method comprising:
administering to the subject an effective amount of a nitrated fatty acid compound selected from the group consisting of NO₂-cLA, CP-36, CP-45, CP-46, CP-51, and analogues/derivatives thereof.

16. The method of claim 15, wherein the nitrated fatty acid compound is CP-36.

17. The method of claim 15, further comprising administering to the subject one or more analgesic agents.

18. A method for the treatment of endometriosis in a subject, the method comprising:
administering to the subject an effective amount of a nitrated fatty acid compound selected from the group consisting of NO₂-cLA, CP-36, CP-45, CP-46, CP-51, and analogues/derivatives thereof.

19. The method of claim 18, wherein the nitrated fatty acid compound is CP-36.

20. The method of claim 18, further comprising administering to the subject one or more analgesic agents.
